(19) United States
(12) Patent Application Publication
(10) Pub. No.: US 2019/0105097 A1
(43) Pub. Date: Apr. 11, 2019

(54) METHODS AND APPARATUS FOR TATTOO REMOVAL

(71) Applicants: Thanh X. Nguyen, Fremont, CA (US); Tam Luu, Fremont, CA (US)

(72) Inventors: Thanh X. Nguyen, Fremont, CA (US); Tam Luu, Fremont, CA (US)

(21) Appl. No.: 15/730,358

(22) Filed: Oct. 11, 2017

(51) Int. Cl. A61B 18/14; A61N 1/06

(52) U.S. Cl. CPC A61B 18/14; A61B 2017/00769; A61N 1/06

(57) ABSTRACT

An apparatus for removing a tattoo includes a control unit having one or more power sources; a head unit coupled to the control unit, wherein the head unit includes a body and a plurality of needles protruding from the body, wherein the plurality of needles includes at least one supply needle and at least one return needle; and a flexible tube coupling the head unit to the control unit, wherein the one or more power sources are electrically coupled to the plurality of needles through the flexible tube, wherein the control unit is configured to deliver power to the at least one supply needle such that power flows from the at least one supply needle to the at least one return needle during use.

### FIELD

Embodiments of the present disclosure generally relate to methods and apparatus for the removal of tattoos.

### BACKGROUND

Tattoo removal procedures are increasing in popularity. Presently, tattoo removal procedures include laser removal, dermabrasion, TCA (trichloroacetic acid, an acid that removes the top layers of skin, reaching as deep as the layer in which the tattoo ink resides), salabrasion (scrubbing the skin with salt), cryosurgery, and excision, which is sometimes still used along with skin grafts for larger tattoos. However, all of these methods fail to completely remove the tattoo and often leave some form of scarring. Laser removal is currently the best method for tattoo removal, but also has a drawback. It often creates blistering due to the burning of the top layer of the skin to penetrate down to the tattoo.

Accordingly, the inventors have devised improved methods and apparatus for removing a tattoo.

### SUMMARY

Methods and apparatus for removing a tattoo are provided herein. In some embodiments, an apparatus for removing a tattoo includes a control unit having one or more power sources; a head unit coupled to the control unit, wherein the head unit includes a body and a plurality of needles protruding from the body, wherein the plurality of needles includes at least one supply needle and at least one return needle; and a flexible tube coupling the head unit to the control unit, wherein the one or more power sources are electrically coupled to the plurality of needles through the flexible tube, wherein the control unit is configured to deliver power to the at least one supply needle such that power flows from the at least one supply needle to the at least one return needle during use.

In some embodiments, a method of removing a tattoo includes placing a head unit of an apparatus for removing the tattoo onto a first area of skin above a first portion of the tattoo; and delivering power to at least one supply needle of the head unit to heat ink, disposed between the at least one supply needle and at least one adjacent return needle, sufficiently to damage ink forming the tattoo.

In some embodiments, an apparatus for removing a tattoo includes a control unit having one or more RF power sources; a head unit coupled to the control unit, wherein the head unit includes a body and a plurality of needles protruding from the body, wherein the plurality of needles includes at least on supply needle and at least one return needle, and wherein each of the plurality of needles extends between about 1 mm and 2 mm beyond a surface of the body; and a flexible tube coupling the head unit to the control unit, wherein the one or more RF power sources are electrically coupled to the plurality of needles through the flexible tube, wherein the control unit is configured to deliver RF power to the at least one supply needle such that power flows from the at least one supply needle to the at least one return needle during use.

Other and further embodiments of the present disclosure are described below.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 depicts a schematic view of an apparatus for removing a tattoo in accordance with some embodiments of the present disclosure.

FIG. 2 depicts a schematic bottom view of a head unit of an apparatus for removing a tattoo in accordance with some embodiments of the present disclosure.

FIG. 3 is a flowchart illustrating a method of removing a tattoo in accordance with some embodiments of the present disclosure.

To facilitate understanding, identical reference numerals have been used, where possible, to designate identical elements that are common to the figures. The figures are not drawn to scale and may be simplified for clarity. Elements and features of one embodiment may be beneficially incorporated in other embodiments without further recitation.

### DETAILED DESCRIPTION

Embodiments of methods and apparatus for removing a tattoo are provided herein. The inventive methods and apparatus advantageously remove the tattoo by directly targeting the ink to be removed.

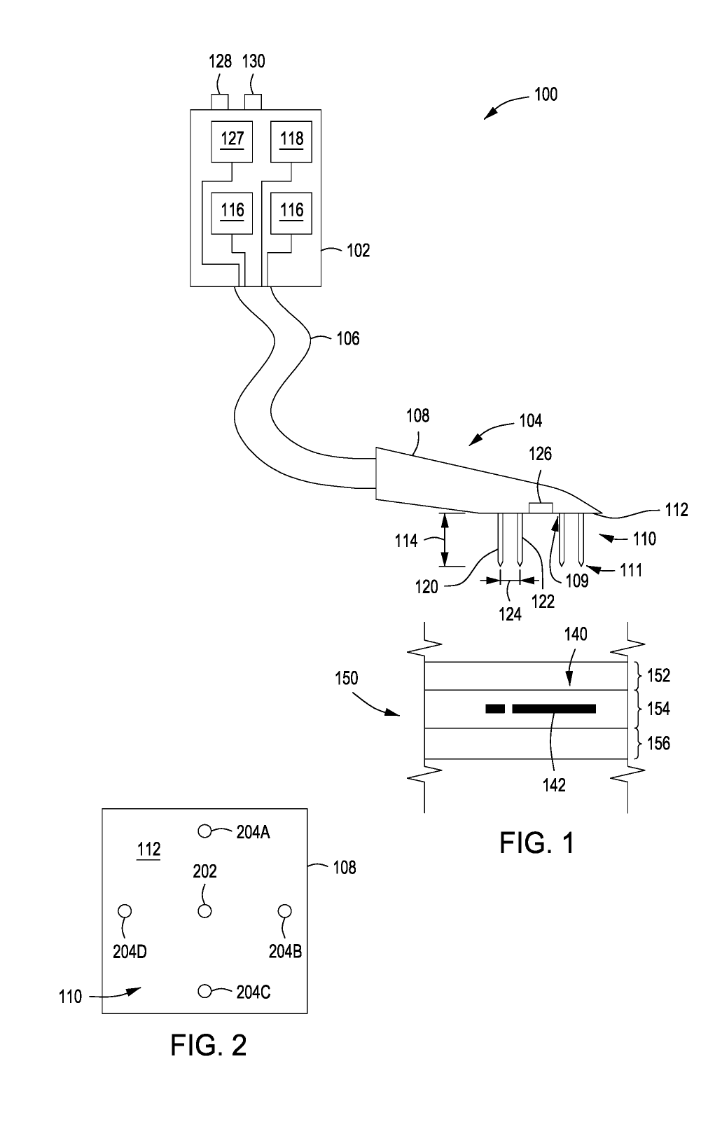

FIG. 1 depicts a schematic view of an apparatus 100 for removing a tattoo 140 in accordance with some embodiments of the present disclosure. In some embodiments, the apparatus 100 includes a control unit 102, a head unit 104, and one or more flexible tubes 106 coupling the head unit 104 to the control unit 102.

The head unit 104 includes a body 108 and a plurality of needles 110 protruding from the body. For example, the plurality of needles 110 may protrude from a surface 112 of the body 108, such as a lowermost surface. Each of the plurality of needles 110 includes a proximate end 109 proximate the body 108 and a distal end 111 disposed furthest away from the body 108. The plurality of needles 110 are at least partially fabricated from a conductive material to provide a conductive path to the distal end 111 of each needle 110 as discussed in more detail below. In some embodiments, the plurality of needles 110 may be formed of stainless steel. Alternatively, the plurality of needles 110 may be formed of any electrically conductive material of sufficient strength for use as described herein.

The plurality of needles 110 may be coupled to the body 108 via any suitable means. For example, the plurality of needles 110 may be inserted into corresponding holes (not shown) in the body 108 containing retaining clamping elements to facilitate coupling and decoupling of the plurality of needles 110 to and from the body 108. In some embodiments, the head unit 104 may be disposable and the plurality of needles 110 may alternatively be permanently coupled to the body 108. In some embodiments, the plurality of needles 110 may be extendable and retractable such that the plurality of needles 110 are at least partially disposed, or in some embodiments completely disposed, within the body 108 when not in use.

In some embodiments, the plurality of needles 110 includes between 2 and 16 needles. The needles may have any suitable diameter for piercing the skin of a person in a tattoo removal process as discussed herein. In some embodiments, each of the plurality of needles 110 may have a diameter between about 0.01 mm and about 0.05 mm. In some embodiments, the plurality of needles 110 may be contained within 1 square inch.

As illustrated in FIG. 1, human skin 150 consists of three layers: the epidermis layer 152, the dermis layer 154 disposed beneath the epidermis layer 152, and the hypodermis layer 156 disposed beneath the dermis layer 154. The epidermis layer 152 and the dermis layer 154 are typically each about 1 mm thick. As such, in some embodiments, each of the plurality of needles 110 may extend beyond the surface 112 by a first distance 114 between about 1 mm and about 2 mm to ensure that a distal end of each of the plurality of needles is adjacent ink 142 that forms the tattoo. In some embodiments, different ones of the plurality of needles 110 may alternatively extend beyond the surface 112 by varying distances (not shown). As the thickness of the human skin can vary from person to person, the first distance 114 can also be greater 2 mm or less than 1 mm.

In some embodiments, the control unit 102 houses one or more power sources 116 which are electrically coupled to the plurality of needles 110 through the one or more flexible tubes 106. In some embodiments, the one or more power sources 116 may be radio frequency (RF) power sources configured to deliver power to the plurality of needles 110 having a magnitude between about 0.25 Watts and about 50 Watts at a frequency between about 25 Hz and about 250 KHz. In some embodiments, the one or more power sources 116 may alternatively be direct current (DC) power sources configured to deliver a similar magnitude of continuous or pulsed power to the plurality of needles 110. In some embodiments, the control unit 102 may also include a coolant source 118 configured to supply a coolant such as, for example, water, to the head unit 104 through the one or more flexible tubes 106 to maintain the head unit at a predetermined temperature between about 70° F. and about 90° F. In some embodiments, the control unit 102 may be configured to continuously supply the coolant to the head unit 104. In some embodiments, the control unit 102 may alternatively be configured to supply the coolant to the head unit at predetermined time intervals. In some embodiments, the head unit 104 may include a temperature sensor (not shown) that signals the control unit to begin supplying coolant to the head unit 104 once a predetermined temperature is reached (e.g., the head unit 104 reaches or exceeds the predetermined temperature) and, optionally, to stop supplying coolant to the head unit 104 once a second predetermined temperature is reached (e.g., the head unit 104 is cooled sufficiently to reach or go below the second predetermined temperature).

The plurality of needles 110 includes at least one supply needle 120 and at least one adjacent return needle 122. Each supply needle 120 is coupled to one or more power sources 116. Each return needle 122 is coupled to ground. The ground may be any suitable ground connection, such as a ground of any of the power sources 116. In some embodiments, a distance between adjacent ones of the plurality of needles (i.e., the target area 124) is between about 0.04 inches and about 0.06 inches. In some embodiments, one supply needle 120 may be paired to a single return needle 122 in a one-to-one relationship, where each pair defines a power flow path. In some embodiments, one supply needle 120 may alternatively be paired to a plurality of return needles 122, where the number of return needles 122 defines the number of power flow paths. In some embodiments, the control unit 102 may include one power source 116 for every power flow path between a supply needle 120 and a return needle 122. That is, a separate power source 116 exists for each power flow path defined by a supply needle 120 and a return needle 122. In embodiments in which the plurality of needles 110 includes between 2 and 16 needles (e.g., 1-8 pairs of supply and return needles 120, 122), the plurality of power sources 116 may include between 1 and 8 power sources, each one corresponding to a distinct pair of supply and return needles 120, 122. For example, as depicted in FIG. 1, the plurality of needles 110 may include two supply needles 120 and two corresponding return needles 122. In embodiments with one supply needle 120 and a plurality of return needles 122, the control unit 102 may include one power source 116 or, alternatively, a plurality of power sources corresponding to the plurality of power flow paths (e.g., one power source per flow path). Alternatively, any one or more of the power sources 116 may provide power to a plurality of the defined flow paths.

In operation, power is supplied to each supply needle 120 from one or more of the one or more power sources 116; electrical current travels from the supply needle to the return needles 122 through the skin; and the electrical current returns to ground via the return needle 122. As a result, a portion of the tattoo 140 disposed within a target area 124 between each set of supply and return needles 120, 122 is heated by the resulting electrical current flowing through the power flow path from the supply needle 120 through the skin and to the return needle 122.

In some embodiments, the head unit 104 may further include a visual monitoring device 126 such as, for example, a camera or a fiber optic visualization device disposed through the surface 112 to display an area beneath the head unit 104. For example, the control unit 102 may include a display device 127 (e.g., a display monitor) electrically coupled to the visual monitoring device 126 to display the area beneath the head unit 104. In some embodiments, the control unit 102 may additionally include one or more buttons, sliders, and/or control knobs 128, 130 (two shown in FIG. 1) to facilitate control of any of the plurality of power sources 116, the coolant source 118, and/or the display device.

FIG. 2 depicts a schematic bottom view of the surface 112 in accordance with some embodiments of the present disclosure. As illustrated in FIG. 2, the plurality of needles 110 may include one supply needle 202 and four return needles 204A-D to facilitate four power flow paths from the supply needle 202 to respective ones of the four return needles 204A-D. In some embodiments including one supply needle and four return needles as depicted in FIG. 2, the control unit may include four power sources 116 (i.e., one power source corresponding to each power flow path). However, the embodiment depicted in FIG. 2 is exemplary and any configuration having power flowing from one or more supply needles to one or more return needles is possible.

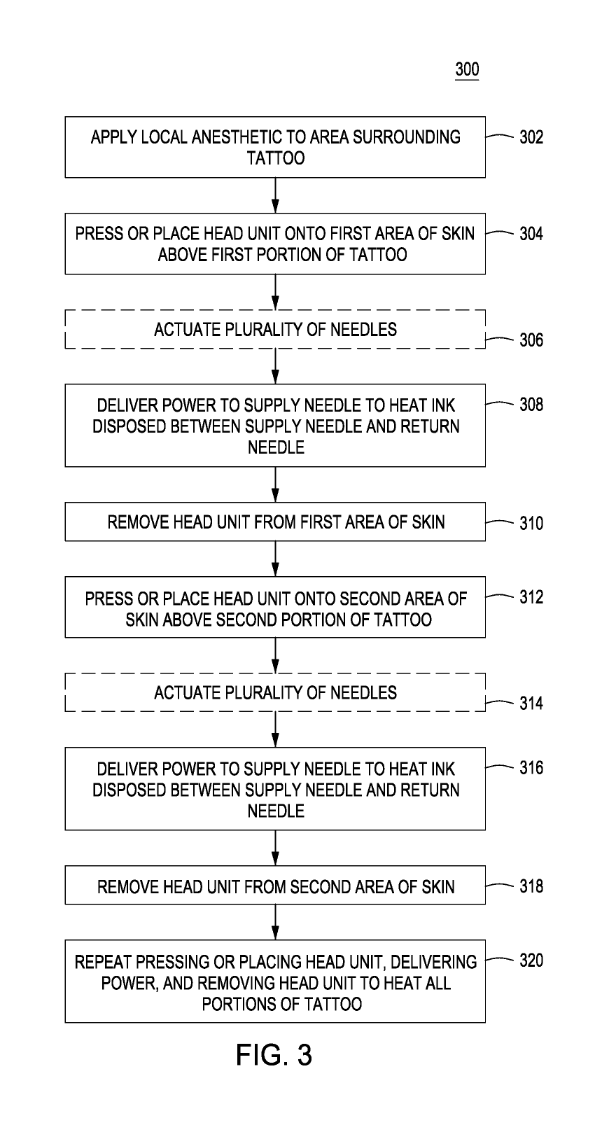

FIG. 3 is a flowchart illustrating a method 300 of removing a tattoo in accordance with some embodiments of the present disclosure. The method 300 begins at 302, where a local anesthetic is applied to an area surrounding the tattoo 140. At 304, the head unit 104 is placed onto a first area of the skin 150 above a first portion of the tattoo 140. In embodiments in which the plurality of needles 110 are not retractable, the head unit is pressed onto the first area of the skin 150 at 304 so that the plurality of needles 110 extend into the first area of skin 150 such that a distal end 111 of each of the plurality of needles 110 extends into the first portion of the tattoo 140. For example, the distal ends 111 of the plurality of needles 110 may be pressed into the skin sufficiently such that the distal ends 111 are positioned proximate to the layer of ink forming the tattoo 140. In embodiments in which the plurality of needles 110 are retractable, at 306, the plurality of needles 110 are actuated so that they puncture the skin and the plurality of needles 110 extend into the first area of skin 150 such that, as discussed above, a distal end 111 of each of the plurality of needles 110 extends into the first portion of the tattoo 140. At 308, power is delivered from one or more power sources 116 to the supply needle 120 to heat ink 142 disposed between the supply needle 120 and the return needle 122 (i.e., within the target area 124). At 310, the head unit 104 is removed from the first area of the skin.

At 312, the head unit 104 is placed onto a second area of the skin 150 above a second portion of the tattoo 140. In embodiments in which the plurality of needles 110 are not retractable, the head unit it pressed onto the second area of the skin 150 at 304 so that the plurality of needles 110 extend into the second area of skin 150 such that a distal end 111 of each of the plurality of needles 110 extends into the second portion of the tattoo 140. In embodiments in which the plurality of needles 110 are retractable, at 314, the plurality of needles 110 are actuated so that they puncture the skin and the plurality of needles 110 extend into the second area of skin 150 such that a distal end 111 of each of the plurality of needles 110 extends into the second portion of the tattoo 140. At 316, power is delivered from the one or more power sources 116 to the supply needle 120 to heat ink 142 disposed between the supply needle 120 and the return needle (i.e., within the target area 124). At 318, the head unit 104 is removed from the second area of the skin 150. At 320, the placing/pressing of the head unit 104 onto another area of the skin 150 above another portion of the tattoo 140, the optional actuating of the retractable needles, the delivering of power to the plurality of needles 110, and the removing of the head unit 104 are repeated until all portions of the tattoo have been heated to damage the ink 142 defining the tattoo 140. Afterwards, the person's body naturally flushes out the damaged ink.

In some embodiments, the power delivered at 306 and 312 (and any other time power is delivered to heat the ink) is RF power having a magnitude between about 0.25 Watts and about 50 Watts and a frequency between about 25 Hz and about 250 KHz. In some embodiments, the RF power is delivered for a period between about 1 second and about 4 seconds.

In some embodiments, the method 300 further includes flowing a coolant from the coolant source 118 to the head unit 104 maintain the head unit 104 at a temperature between about 70° F. and 90° F. In some embodiments, the coolant is flowed to the head unit 104 continuously.

While the foregoing is directed to embodiments of the present disclosure, other and further embodiments of the disclosure may be devised without departing from the basic scope thereof.

1. An apparatus for removing a tattoo, comprising:
a control unit having one or more power sources;
a head unit coupled to the control unit, wherein the head unit includes a body and a plurality of needles protruding from the body, wherein the plurality of needles includes at least one supply needle and at least one return needle; and
a flexible tube coupling the head unit to the control unit, wherein the one or more power sources are electrically coupled to the plurality of needles through the flexible tube,
wherein the control unit is configured to deliver power to the at least one supply needle such that power flows from the at least one supply needle to the at least one return needle during use.

2. The apparatus of claim 1, wherein the one or more power sources are radio frequency (RF) power sources configured to deliver RF power having a magnitude between about 0.25 Watts and about 50 Watts and a frequency between about 25 Hz and about 25 KHz to the at least one supply needle.

3. The apparatus of claim 1, wherein the one or more power sources are direct current (DC) power sources configured to deliver pulsed or continuous DC power to pairs of the plurality of needles.

4. The apparatus of claim 1, wherein the one or more power sources includes one power source for every power flow path between a supply needle and a return needle.

5. The apparatus of claim 1, wherein the plurality of needless is between 2 and 16 needles.

6. The apparatus of claim 5, wherein the one or more power sources includes between 1 and 8 power sources.

7. The apparatus of claim 1, wherein each of the plurality of needles extends between about 1 mm and 2 mm beyond a surface of the body.

8. The apparatus of claim 1, further comprising:
a coolant source disposed in the control unit, wherein the coolant source is fluidly coupled to the head unit through the flexible tube, wherein the coolant source is configured to flow a coolant through the head unit to maintain the head unit at a temperature between about 70° F. and 90° F.

9. The apparatus of claim 1, wherein the plurality of needles are contained within 1 square inch.

10. The apparatus of claim 1, wherein the plurality of needles includes one supply needle and four return needles to facilitate four power flow paths from the supply needle to respective ones of the four return needles.

11. The apparatus of claim 1, wherein a distance between adjacent ones of the plurality of needles is between about 0.04 inches and about 0.6 inches.

12. The apparatus of claim 1, wherein the head unit further comprises a visual monitoring device disposed through a surface of the body to display an area beneath the head unit.

13. The apparatus of claim 1, wherein the plurality of needles are formed of stainless steel.

14. A method of removing a tattoo, comprising:
placing a head unit of an apparatus for removing the tattoo onto a first area of skin above a first portion of the tattoo; and
delivering power to at least one supply needle of the head unit to heat ink, disposed between the at least one supply needle and at least one adjacent return needle, sufficiently to damage ink forming the tattoo.

15. The method of claim 14, where the power delivered is RF power having a magnitude between about 0.25 Watts and about 50 Watts and a frequency between about 25 Hz and about 250 KHz.

16. The method of claim 14, wherein the power is delivered for a period between about 1 second and about 4 seconds.

17. The method of claim 14, further comprising:
flowing a coolant from a coolant source to the head unit to maintain the head unit at a temperature between about 70° F. and 90° F.

18. The method of claim 17, wherein the coolant is continuously flowed to the head unit.

19. An apparatus for removing a tattoo, comprising:
a control unit having one or more RF power sources;
a head unit coupled to the control unit, wherein the head unit includes a body and a plurality of needles protruding from the body, wherein the plurality of needles includes at least on supply needle and at least one return needle, and wherein each of the plurality of needles extends between about 1 mm and 2 mm beyond a surface of the body; and
a flexible tube coupling the head unit to the control unit, wherein the one or more RF power sources are electrically coupled to the plurality of needles through the flexible tube,
wherein the control unit is configured to deliver RF power to the at least one supply needle such that power flows from the at least one supply needle to the at least one return needle during use.

20. The apparatus of claim 19, further comprising:
a coolant source disposed in the control unit, wherein the coolant source is fluidly coupled to the head unit through the flexible tube, wherein the coolant source is configured to flow a coolant through the head unit to maintain the head unit at a temperature between about 70° F. and 90° F.
